(19) United States
(12) Patent Application Publication
(10) Pub. No.: US 2025/0242170 A1
(43) Pub. Date: Jul. 31, 2025

(54) Methods and Systems for Improving Photodynamic Therapy by Increasing Depth of Penetration Without Concurrently Increasing Energy Deposition

(71) Applicant: Todd Frank Ovokaitys, Carlsbad, CA (US)

(72) Inventor: Todd Frank Ovokaitys, Carlsbad, CA (US)

(21) Appl. No.: 19/036,489

(22) Filed: Jan. 24, 2025

Related U.S. Application Data
(60) Provisional application No. 63/624,859, filed on Jan. 25, 2024.

(51) Int. Cl. A61N 5/06; A61K 35/545; A61K 41/00; A61P 35/00

(52) U.S. Cl. CPC A61N 5/062; A61K 35/545; A61K 41/0057; A61P 35/00; A61N 2005/0658

(57) ABSTRACT

Methods and systems are provided for treating a predefined disease in a patient characterized by neoplastic malignancy. Accordingly, a dose of a photoactive compound is administered to the patient using a delivery method, followed by waiting for a predetermined period of time. An area of the patient is treated with modulated pulses of laser light having a predefined wavelength and for another predefined period of time.

### CROSS-REFERENCE

The present application relies on U.S. Patent Provisional Application No. 63/624,859, titled “Methods and Systems for Improving Photodynamic Therapy by Increasing Depth of Penetration without a Concurrent Unacceptable Increase in Energy Deposition” and filed on Jan. 25, 2024, for priority.

### FIELD

The present specification discloses methods and systems for improving photodynamic therapy, relative to conventional approaches, by enabling an increase in a therapeutic depth of penetration without having the patient experience an increase in energy deposition above a predefined level from the point of skin illumination to the therapeutic site.

### BACKGROUND

Photodynamic Therapy (PDT) is a method for treating neoplasias that are superficially located or present at a shallow distance (for example, 5 mm or less) from the surface of a tissue or organ. Well known in this art is a porfimer sodium solution, known as Photofrin®, the use of which is exemplary for this therapeutic process. Photofrin® was approved in Canada in 1993 for the treatment of bladder cancer. Photofrin® was approved by the US FDA in 1995 for esophageal cancer and in 1998 for early-stage non-small cell lung cancer (NSCLC). The treatment protocol for esophageal or NSCLC includes, as a first step, administering a single dose of Photofrin® (2 mg/Kg) as a slow IV push over a duration of 3-5 minutes. After administration, Photofrin® is absorbed by body tissues, including the neoplastic cells. Once the waiting period of approximately 40-50 hours is completed, the Photofrin® is largely eliminated from healthy cells, and is relatively concentrated in the abnormal cells. Photofrin® remains concentrated in the abnormal cells and is eliminated from the normal cells because the normal cells have enough aerobic energy to effectuate removal of the compound (Photofrin®). On the other hand, the neoplastic cells do not have the energy to push out the compound as they are have considerably lower energy. The waiting period allows for concentration of the compound in the abnormal cells. As is known in the art, a normal cell produces 36 ATP molecules per molecule of glucose. In comparison, an abnormal cell, such as a malignant cell, is anaerobic and only produces 2 ATP molecules per molecule of glucose. In addition abnormal cells have a reduced cellular voltage and therefore potential energy.

The second step includes applying 630 nm laser light typically on the order of 2000 mW (or 2 Watts) intensity directly to the involved tissue through a fiberoptic channel of an endoscope or bronchoscope after the 40-50 hour waiting period. For esophageal cancer the duration of laser light application is 8 minutes and 20 seconds. For NSCLC the duration is 12 minutes and 30 seconds. Upon application of the 630 nm laser light, the Photofrin® is activated in the abnormal cells in a reaction that destroys the abnormal cells while leaving the normal cells largely unharmed.

The general method of photodynamic therapy (PDT) is thus to administer a photoactive compound and provide compound-dependent, predetermined period of time for the compound to clear from normal cells and be relatively more concentrated in the abnormal cells. Then laser light is applied to eliminate the abnormal cells.

However, current PDT methods are limited by the depth of penetration of the laser light used to activate the photoactive compounds and destroy the abnormal cells. The depth of penetration for visible light (400 nm to 700 nm) is 2-3 mm in the blue violet spectrum (with wavelengths ranging from 380 nm to 495 nm) and 4-5 mm in the red end of the spectrum (620 nm to 750 nm).

Thus, what is also needed is a method to ensure a significant anti-neoplastic effect at an appropriate depth penetration of the laser light to treat various conditions that may have abnormal cell activity.

### SUMMARY

The following embodiments and aspects thereof are described and illustrated in conjunction with systems, tools and methods, which are meant to be exemplary and illustrative, not limiting in scope. The present specification discloses numerous embodiments.

In embodiments, the present specification is directed towards a method of treating a predefined disease in a patient characterized by a neoplastic malignancy, the method comprising: administering a photoactive compound to the patient; waiting for a first period of time; and immediately after the first period of time, applying modulated pulses of a laser light beam having a predefined wavelength to an area of the patient for a second period of time, wherein the modulated pulses of the laser light beam are formed by passing the laser light beam from the laser through a phase cancellation optical element, wherein the phase cancellation optical element is adapted to form a pattern of constructive interference nodes and destructive interference nodes, and wherein a depth of penetration of the laser light beam in the patient is increased after the administration of the photoactive compound to the patient relative to a depth of penetration of the laser light beam in the patient without the administration of the photoactive compound.

Optionally, the phase cancellation optical element comprises a first diffraction grating, a refractive element and a second diffraction grating positioned in series.

Optionally, the laser is configured to form a Fresnel zone in the laser light beam.

Optionally, the photoactive compound is a dye. Optionally, the photoactive compound is a porfimer sodium solution.

Optionally, the first period of time is in a range of 2 days to 5 days.

Optionally, the photoactive compound is nanoscale and microencapsulated Indocyanine Green (ICG). Optionally, the photoactive compound is Methylene Blue.

Optionally, a depth of penetration of the laser light beam in the patient is increased after the administration of the photoactive compound to the patient by a factor of 10% to 3000% relative to a depth of penetration of the laser light beam in the patient without the administration of the photoactive compound.

Optionally, the laser light beam has a primary beam power of 3 W to 7 W.

Optionally, the predefined wavelength ranges from 300 nm to 1000 nm and is dependent on the photoactive compound that is used.

Optionally, the predefined wavelength is either equal to or below 400 nm or equal to or above 700 nm. Optionally, the predefined wavelength is 810 nm. Optionally, the predefined wavelength is 660 nm.

Optionally, the method further comprises administering VSEL stem cells to the patient. Optionally, the method comprises activating VSEL stem cells prior to said administration by exposing the VSEL stems cells to modulated laser light. Optionally, the method further comprises activating VSEL stem cells after said administration by exposing the VSEL stems cells in vitro to modulated laser light.

Optionally, the method further comprises reevaluating the patient's neoplastic malignancy to determine an improvement in said malignancy by at least either a reduction in size or a reduction in a rate of growth. Optionally, the method further comprises repeating the administration of the photoactive compound, waiting for the first period of time, and application of modulated pulses of laser light if the patient's neoplastic malignancy has not shown a reduction in size or rate of growth level of at least 5%.

In embodiments, the present specification is directed towards a kit for treating a predefined disease in a patient characterized by a neoplastic malignancy, comprising: a photoactive compound, wherein the photoactive compound is at least one of a dye, Photofrin, Indocyanine Green, and Methylene Blue; and a laser system adapted to emit laser light having a wavelength in a range of 300 nm to 1000 nm, wherein the laser system is configured to pass a laser beam through a phase cancellation optical element, wherein the phase cancellation optical element is adapted to form a pattern of constructive interference nodes and destructive interference nodes, and wherein the laser beam has a power in a range of 3 W to 7 W.

Optionally, the laser system is configured to form the laser beam such that a depth of penetration of the laser beam in the patient is increased after the administration of the photoactive compound to the patient relative to a depth of penetration of the laser beam in the patient without the administration of the photoactive compound.

Optionally, the laser system is configured to form the laser beam such that a depth of penetration of the laser beam in the patient is increased by a factor of 10% to 3000% after the administration of the photoactive compound to the patient relative to a depth of penetration of the laser beam in the patient without the administration of the photoactive compound.

Optionally, the phase cancellation optical element comprises a first diffraction grating, a refractive element and a second diffraction grating positioned in series.

Optionally, the laser system is configured to form a Fresnel zone in the laser beam.

Optionally, the laser beam has a beam power of 5 W.

Optionally, the wavelength of the laser light is 810 nm. Optionally, the wavelength of the laser light is 660 nm.

In embodiments, the present specification is directed towards a method of treating a predefined disease in a patient characterized by a neoplastic malignancy, the method comprising: administering a photoactive compound to the patient in a dose, using a delivery method, and waiting for a first predetermined period of time; and, treating an area of the patient with modulated pulses of laser light having a predefined wavelength and for a second predefined period of time.

Optionally, the photoactive compound is a dye. Optionally, the photoactive compound is Photofrin and the first predetermined period of time is in a range of 2-5 days, preferably 3 days. Still optionally, the photoactive compound is nanoscale and microencapsulated Indocyanine Green (ICG). Still optionally, the photoactive compound is Methylene Blue.

Optionally, the laser light is selected such that the depth of penetration is increased while the power/energy deposition is decreased. Optionally, the depth of penetration of the laser light is increased by a factor of 10% to 3000%. Optionally, the laser light has a primary beam power of 5 W.

Optionally, the predefined wavelength ranges from 300 nm to 1000 nm and is dependent on the photoactive compound that is used. Optionally, when Photofrin is used, the predefined wavelength is 630 nm. Optionally, where ICG is used, the predefined wavelength is 810 nm. Optionally, where Methylene Blue is used, the predefined wavelength is 660 nm.

Optionally, laser-activated VSEL stem cells are administered to the patient. Optionally, a SONG modulated laser light is used to activate VSEL stem cells. Optionally, the SONG modulated laser light is applied to stem cells in vitro.

Optionally, the photoactive compound is administered intravenously.

Optionally, the method further comprises reevaluating the patient's neoplastic malignancy for determining improvement in said malignancy by at least either a reduction in size or a reduction in a rate of growth. Optionally, the method still further comprises repeating at predefined intervals of time the administration of the treatment methods to the patient if the patient's neoplastic malignancy has not shown a reduction in size or rate of growth level; and concluding the administration of the treatment methods if the patient's neoplastic malignancy has shown a significant reduction in size or rate of growth level.

The aforementioned and other embodiments of the present shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1 illustrates a Strachan-Ovokaitys Node Generator (SONG) device as disclosed in U.S. Pat. No. 6,811,564, which is incorporated herein by reference in its entirety;

FIG. 2 shows a sparse constructive interference effect from a 1 percent bandwidth cancellation plate having a 5 mm aperture;

FIG. 3 is a flow chart illustrating an exemplary process of using the combination of PDT with a SONG optics treated laser, in accordance with some embodiments of the present specification;

FIG. 4 is a flow chart illustrating an exemplary process of treatment using Photofrin® in conjunction with the SONG optics treated laser, in accordance with some embodiments of the present specification;

FIG. 5 is a flow chart illustrating another exemplary process using Methylene Blue, in conjunction with the SONG optics treated laser in accordance with some embodiments of the present specification; and

FIG. 6 is a block diagram showing a kit which includes at least one photoactive compound and a laser system, in accordance with some embodiments of the present specification.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise” “include” and “have”, and forms thereof, are not necessarily limited to members in a list with which the words may be associated. It should be noted herein that any feature or component described in association with a specific embodiment may be used and implemented with any other embodiment unless clearly indicated otherwise.

### SONG Device

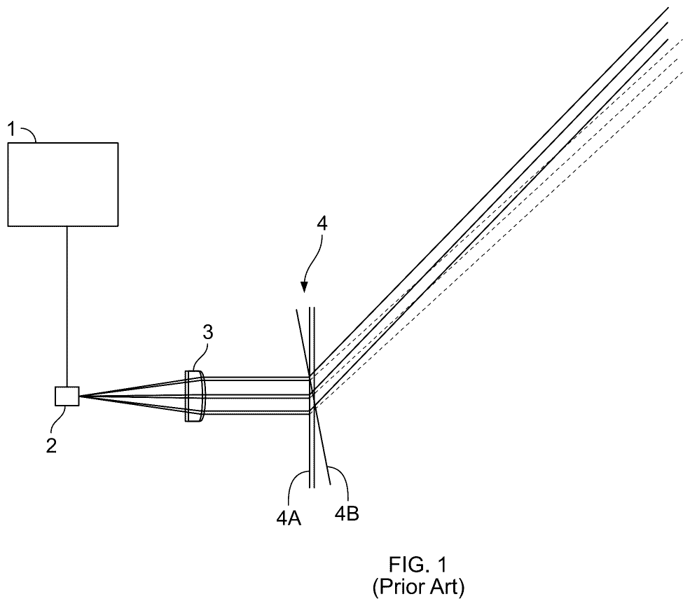

FIG. 1 illustrates a SONG device as disclosed in U.S. Pat. No. 6,811,564, which is incorporated herein by reference. Referring to FIG. 1, the SONG device comprises a laser diode 2 which is controlled by an amplitude modulator 1. The laser diode 2 is selected to have a substantially linear relationship between current and wavelength with minimum mode hopping. The amplitude modulator 1 modulates the current to the laser diode 2 which, in turn, results in a very small wavelength modulation of the laser, for purposes discussed below.

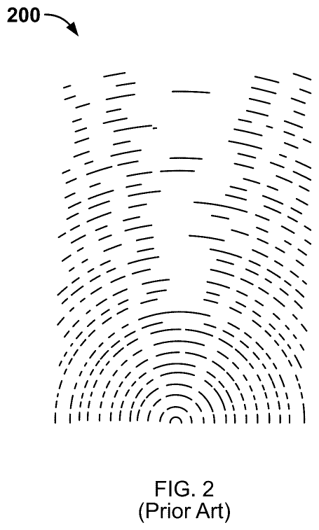

The output of the laser diode 2 is collimated by a lens 3 and passed to an optical element 4. The optical element 4 consists of a first diffraction grating, a refractive element, and a second diffraction grating such that the beam is substantially cancelled. This allows the cancellation to occur over a small percentage of the wavelength variance of the laser source, rather than at a single critical wavelength. Wavelengths beyond the acceptance bandwidth of the cancelling optic 4 above and below the center frequency pass without being cancelled. This means that a complex Fresnel/Fraunhoffer zone is generated, defined by the beat frequency of the high and low frequencies as a function of the aperture. Consequently, relatively sparse zones of constructive interference occur between the high and low frequency passes of the cancellation element in selected directions from the aperture, as shown in FIG. 2. FIG. 2 shows the sparse constructive interference effect from a 1 percent bandwidth cancellation plate of 5 mm aperture. Black represents constructive nodes.

As seen in FIG. 1, the optical element 4 can be adjusted angularly between positions 4A and 4B. This varies the ratio of constructive to destructive interference. Additionally, in embodiments, the system of FIG. 1 may include mechanisms for aligning the resultant beam emerging from optical element 4, in a straight line with the collimated beam emerging from collimator 3.

In effect, the continuous beam is transformed into a string of extremely short duration pulses typically on the order of a duration in subfemtoseconds. The small wavelength modulation of the laser diode 2 causes the constructive and destructive nodes to move rapidly through the volume of the Fresnel zone of the collimator lens aperture. This has the effect of stimulating very short (subpicosecond) pulse behavior at any point in the Fresnel zone through which the nodes pass at a pulse repetition frequency defined by the amplitude modulator frequency.

The wavelength of the cancellation and constructive interference zones for a theoretical single path would be the difference between the two frequencies. If the bandwidth of the cancelling element is narrow, this difference is very small and the effective wavelength of the cancelled/non-cancelled cycle would be very long, on the order of pico-seconds. Therefore, the system would behave substantially similarly to a system with no cancellation because it requires an aperture much larger than the primary light wavelength to generate a useful Fresnel/Fraunhoffer zone. Such an aperture would greatly multiply the available Feynman diagram paths eliminating any useful effect, even if it were possible to generate a sufficiently coherent source of such an aperture.

If the beat frequency can be made high enough, the wavelength of the cancelled to non-cancelled cycle can be a fraction of a practical aperture. This will make this wavelength sufficiently small to limit the Feynman paths to within a cycle or two in free space allowing the Fresnel/Fraunhoffer effect to be apparent. Since the center frequency and spectrum spread of a laser diode is modulated by adjusting the current and/or temperature of the junction, the pattern of the Fresnel/Fraunhoffer zones is varied substantially by very small variations in the wavelength of one or both pass frequencies. Such modulation is produced in the apparatus of FIG. 1 by the amplitude modulator 2.

A conventional coherent or incoherent beam would have high probability paths in the Feynman diagram. These paths would overlap at very low frequencies (kHz) and be of little practical use in the stimulation of molecular resonance. It should be noted however that the phenomena described above is used as a means to multiply the modulation frequency, up to the point where the beam effectively becomes continuous. Thus, by properly selecting the aperture, the region of the beam selected for transmission through the medium, and the modulation frequency, it is possible to cause the constructive nodes to pass across any given point in the beam at frequencies many times higher than the modulation frequency. In ideal conditions, the duration of exposure to a constructive node of any point would be for a period equivalent to a quarter of the duration of a wavelength of the molecular frequency repeated once per cycle. If the wavelength of the laser is chosen to be one easily absorbed by the atomic structures it is desired to induce to resonance, then the beam will efficiently deliver the desired modulation frequency to the desired molecules.

Photodynamic Therapy (PDT) with SONG Laser Treatment

In some embodiments of the present specification, pairing of photo-active compounds used in PDT therapy procedures with the respective appropriate wavelength lasers using SONG, as described above, produces an anti-neoplastic effect, usually with few or no side effects other than the usual precautions for PDT. For this purpose, a photo-active compound is first delivered to a patient in a specific dose, using a specific delivery method, and a specific predetermined wait period of time. The amount of the dose, the delivery method, and the period of time for waiting are based on the photo-active compound used. The photo-active compounds are also known as dyes, in some embodiments. In embodiments, the following parameters are considered when using photodynamic therapy to treat a condition: the photo-active compound, the appropriate dose for the compound, the preferred delivery method; the corresponding range of time for waiting to clear normal cells; the distinctive color characteristic; the distinctive absorption spectrum of the compound; the frequency and wavelength of light for oxidation reaction; the range of power of the laser source; and the range of depth of penetration. In embodiments, the laser light is selected such that the depth of penetration is increased while the power/energy deposition is decreased.

The photo-active compounds or dyes are photosensitive and therefore any type of exposure towards light after administration, especially infrared (IR), is avoided. Examples of the dyes include Indocyanine Green (ICG) and Methylene Blue. Embodiments of the present specification that use ICG may prepare or formulate the ICG in one or more variations. In one exemplary method, the ICG is in the form of a nanoscale and microencapsulated preparation. In an exemplary case, the ICG accumulates over three consecutive days in the low energy neoplastic cells and requires the same number of days to clear the normal cells. Each photo-active compound has a distinctive color characteristic, a distinctive absorption spectrum, a unique frequency, and color or wavelength of light that can cause an oxidation reaction for the purposes of PDT.

In embodiments, PDT process, when coupled with the SONG laser is used to selectively disintegrate (“melt away”) neoplastic cells, while leaving normal cells unharmed. In an optional embodiment, the natural killer cells (NKCs) and the immune system are then modulated using laser activated VSELs to cause them to increase their efficacy in clearing, destroying and/or removing neoplastic cells. NKCs are adapted to identify cells that express MAC Class 1 and then eliminate them, either through gamma interferon or through cell-to-cell toxicity. Modulating NKCs, in accordance with the present specification, enables the NKCs to more effectively function as an anti-tumor mechanism. NKCs increase gamma interferon in the presence of activated VSELs.

Conventional PDT methods employ laser beams at a higher power level to achieve greater depth of penetration. The current depth of penetration is a function of the fact that, to safeguard human tissue, the energy of the penetrating light must be limited and therefore the depth of penetration is limited. However, such an application of laser energy is likely to result in intense scattering on the skin surface. In an exemplary scenario, a 20 W laser (used without SONG optics in accordance with the present specification), would burn the skin while having the concurrent disadvantage of rapidly decreasing in penetration with each millimeter of depth through the tissue, therefore resulting on rapid fall of the power density. Using a 5000 mW laser at the surface with an ordinary laser (such as for example, a red laser) would result in large diminishing of power density after just 5 mm of depth into skin tissue. Thus, the photons are unable to penetrate deep enough for a successful treatment or therapy.

In embodiments, a beam of a laser source (such as an Ultron™) is transmitted through a Strachan-Ovokaitys Node Generator (SONG) laser device to create a therapeutic laser light beam capable of achieving far greater depth of penetration without an unacceptable increase in power or energy deposition into tissue. Using a SONG laser device in combination with PDT methods, as described in the present specification, improves a depth of penetration of the light beam by 10% to 3000% relative to conventional PDT methods. Using the combination with SONG laser modifies the wave-phase relationships of the beam output from the SONG device, to reduce scatter pathways and drive sparse nodes of constructive interference deeply into tissue.

Additionally, a longer wavelength in the near infra-red spectrum is used, as it has an optical window such that the photons themselves penetrate much more deeply. An example is 810 nm infra-red that is known to penetrate through skin, skull, and into the outer 3-5 cm of brain matter. With this method, bulk photons may be delivered to any part of the brain. A SONG Laser device configured for this wavelength enables these photons to penetrate to any location in the brain and almost any location within any other body or tissue structure.

Another benefit of using the combination of PDT with SONG laser (as opposed to conventional laser) in embodiments of the present specification is that the SONG laser is delivered in such a manner that the surface of the skin is not heated. Once the laser beam traverses through the SONG optics device, the laser beam is reduced to an energy of 1400 milliwatts distributed over a 25 mm diameter. The power dose is small enough such that the surface of skin does not get heated or is not subject to laser burns. Furthermore, therapy and treatment methods of the present specification are non-invasive.

Thus, embodiments of the present specification greatly reduce the scatter pathways compared with conventional lasers, as described above, thereby enabling effective delivery of photons. Also, the power of the laser beam that is output from the SONG optics device and focused on the skin surface is reduced. The use of SONG optics allows use of a higher primary power of the source laser safely, without resulting in burns. In embodiments, the use of the SONG optics allows for the use of a higher primary power because of the waveform reconstruction that is enabled. For PDT applications, the quantity of bulk photons allows for the necessary reactions. In embodiments, a laser beam of up to 500 to 600 mW per cm² can be used (a higher power red or infrared laser). In embodiments, the laser power may be up to 3 W with an area of the beam at 4.9 to 5.0 cm², suggesting that the power of the laser can be doubled (60% cancellation). In embodiments, 80% cancellation may be achieved affording a greater increase in the depth of penetration. In embodiments, an increased depth of penetration may be necessitated for a patient in which an area with a tumor may be obfuscated with an increased number of fat cells (i.e. an obese patient). In embodiments, an absolute limiting factor of the laser energy would be thermal injury or burning tissue.

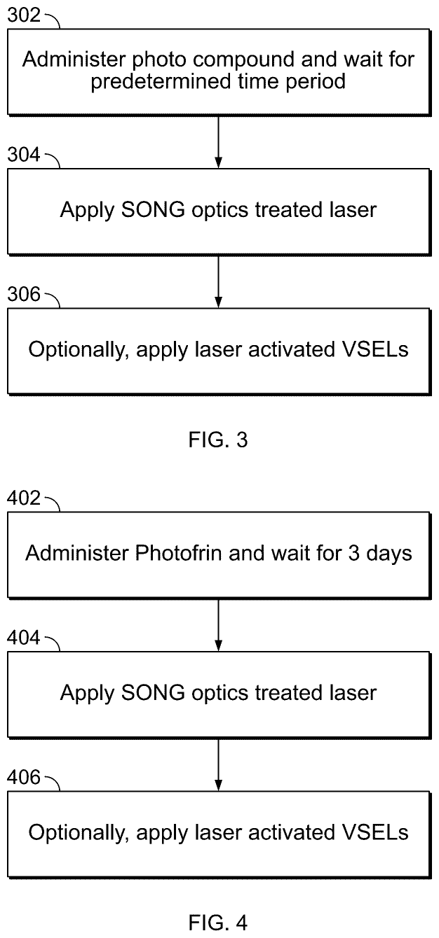

FIG. 3 is a flow chart illustrating an exemplary process of using the combination of PDT with a SONG optics treated laser, in accordance with some embodiments of the present specification. At step 302, a PDT protocol is initiated, comprising administration of a photo-active compound. A first period of time, which, in embodiments is a waiting period, is provided after administration of the photo-active compound during which the photo-active compounds reach all cells and during which normal cells that have sufficient aerobic energy reject the photo-active compound while the neoplastic cells that are abnormal and have low energy are unable to reject the photo-active compound. The waiting period is dependent on the selection of the photo-active compound. Therefore, step 302 is a (photo) compound specific process for selective accumulation and concentration in the cells that are desirable to be destroyed (that is the abnormal, neoplastic cells). In some embodiments, the first waiting period is in a range of two days to five days.

After the first waiting period, and preferably immediately after, at step 304, a laser beam is transmitted through the SONG optics in accordance with the present specification, to generate and subsequently apply light of an appropriate wavelength corresponding to the selected photo-active compound, to stimulate a photo-oxidation reaction. The SONG optics system comprises components that are configured to generate modulated pulses of the laser light beam by passing the laser light beam from the laser through a phase cancellation optical element. The phase cancellation optical element is adapted to form a pattern of constructive interference nodes and destructive interference nodes. Therefore, modulated pulses of the laser light beam are applied to the patient. The laser generation system, also described herein as the SONG optics or SONG optics system, is configured with a first diffraction grating, a refractive element and a second diffraction grating positioned in series. Furthermore, the laser generation system is configured to form a Fresnel zone in the laser light beam.

The laser light beam is applied for a second period of time. The second period of time is less than the first period of time (or a waiting period which commences after administering the photoactive compound). In some embodiments, the wavelength of the applied laser light beam is either equal to or below 400 nanometer (nm) or is equal to or above 700 nm. The selection of the photo-active compound influences the selection of the energy of the source laser beam, the power of the source laser beam, and the wavelength of the source laser beam, to achieve the desirable outcome. In embodiments, the power of the laser beam can range from 3 W to 7 W. The method of FIG. 3 achieves a penetration depth of light that is at least 10% to 3000% more than conventional PDT methods. The depth of penetration ensures that enough bulk photons reach and result in oxidation of neoplastic cells. The treatment method described herein is repeated from steps 302 to 304 if it is observed that the patient's neoplastic malignancy has not shown a reduction in size or rate of growth level of at least 5%.

At step 306, optionally, laser activated VSELs are used to modulate NKCs and the immune system to effectively clear remaining neoplastic cells, while still leaving the normal cells unharmed. It should be noted that VSELs may be activated prior to beginning the PDT and SONG laser therapy of the present specification as a pre-treatment step.

FIG. 4 is a flow chart illustrating another exemplary process using Photofrin® as the photo-active compound, in accordance with some embodiments of the present specification. At step 402, a PDT is initiated, comprising administration of Photofrin®. A 3-day waiting period is provided during which the photo-active compounds contained within Photofrin® reaches the cells within the patient, and during which normal cells that have sufficient aerobic energy reject the photo-active compound, while the neoplastic cells that are abnormal and have low energy are unable to reject the photo-active compound.

At step 404, a laser beam is transmitted through the SONG optics device in accordance with the present specification, to apply light of wavelength of 810 nm (near infra-red) with a primary beam power of 5 W, to stimulate a photo-oxidation reaction. The selection of the photo-active compound influences the selection of energy of source laser beam, the power of the source laser beam, and the wavelength of the source laser beam, to achieve the desirable outcome. The method of FIG. 4 achieves a penetration depth of light that is at least 10% to 3000% more than conventional PDT methods. The depth of penetration ensures that enough bulk photons reach and result in oxidation of neoplastic cells.

At step 406, optionally, laser activated VSELs are used to modulate NKCs and the immune system to clear remaining neoplastic cells, while still leaving the normal cells unharmed. It should be noted that VSELs may be activated prior to beginning the PDT and SONG laser therapy of the present specification as a pre-treatment step.

### Exemplary Use Case Scenarios of PDT Plus SONG Laser

Two exemplary use case scenarios in which a photo-active compound (Photofrin®) is used in conjunction with the SONG Laser will now be described below. Specifically, the usage scenarios describe a treatment protocol for an aggressive brain cancer and a metastatic breast cancer, both of which are considered incurable or with limited likelihood of cure. However, because of the high depth of penetration of the primary wavelength of laser beam and the much greater further depth of penetration imparted by the use of SONG optics device, this method can be used for essentially any primary, secondary or recurrent neoplastic mass of any primary cell type at any location in the body.

Use Case with PDT and SONG Laser in Treatment of Glioblastoma Multiforme

The process is further described within a use case scenario of treating a 55-year-old man diagnosed with glioblastoma multiforme. Glioblastoma multiforme is the most aggressive of the brain cancers characterized by rapid growth and extensions throughout the brain making it surgically incurable. Prognosis without treatment is usually 3 months and with maximum treatment only 18 months. The conventional treatment protocol is neurosurgical debulking, targeted and whole brain radiation, followed by 6 months of chemotherapy with Temodar. After these treatments have been exhausted and disease recurs the prognosis is very poor and short term.

The patient described in the stated use case received debulking neurosurgery, targeted radiation to the areas of visible mass, followed by whole brain radiation, and then 6 months of Temodar. After these treatments he suffered memory loss and emotional instability.

A month after completing his last cycle of chemotherapy, the patient received the SONG Laser-based photodynamic therapy in accordance with the present specification, using ICG as the photo-active compound. On days 1, 2, and 3 of the treatment, the patient received 50 mg of nanoscaled or liposomal ICG. On day 3 he was treated with a SONG laser device at 810 nm near infra-red with a primary beam power of 5 Watts. From the peak power measured after going through the beam expander and SONG generator of 3.5 Watts this was reduced 60% to a measured output power of 1.4 Watts. The treated laser was applied in a beam expanded to 25 mm to the whole brain transcutaneously for 12 minutes total duration, by slowly scanning the 25 mm beam systematically over the skull under which brain substance is located. The next day this protocol with the SONG laser configured the same way was applied to the whole brain, also for 12 minutes total duration. A month later the patient was scanned using MRI, which showed no evidence of recurrence of Glioblastoma multiforme. Over the next 2 months the patient noted improvements in his memory and mood. An MRI scan repeated at this time, 4 months after completing his last chemotherapy, also showed no evidence of recurrence.

In the mentioned use case, an additional adjunct to the patient's PDT treatment included administering SONG Laser activated and guided very small embryonic-like (VSEL) stem cells 3 days before the PDT cycle began. For this process, an IV was started, and 6 tubes of blood of 10 ml each were drawn. Gel separation tubes validated for this purpose were used. These were placed in a centrifuge specified for these tubes that were spun for 10 minutes at 3200 rpm. The centrifugation process removed 99% of red blood cells and over 95% of white blood cells, and provided a preparation of plasma platelets and VSELs, with less than 1% of the VSELs lost in the gel. The product of 3 of the 10 ml tubes, approximately 18-22 ml depending on the hematocrit of the patient, were drawn into a 20 ml or 30 ml syringe depending on total volume. Each of the 2 syringes having the preparation were treated with a SONG Laser for 3 minutes while slowly rotating the plasma up and down through the beam. For this purpose, the laser had a primary power output of 5 mW and a peak output of about 2.5 mW after passing through a beam expander and a SONG optics device. The peak output was further reduced by 60% through adjusting the angle of the optical device to a total ultimate output power of about 1 mW. This beam was applied transcutaneously through the skull by slowly scanning the 25 mm beam through the areas under the skull where brain substance resides. This procedure with SONG Laser activated and guided VSELs was repeated 1 day before the PDT procedure, except with 2.7 times the quantity of VSELs.

Use Case with PDT and SONG Laser in Treatment of Metastatic Breast Cancer

A second case using the protocol as above (described in FIG. 24) was for a 54-year-old woman with recurrent breast cancer in the right breast with metastatic involvement of right axillary lymph nodes, except the optional adjunctive VSEL protocol was performed only once after the enhanced deeply penetrating PDT procedure. A few weeks after her protocol was completed, she underwent surgery for removal of the recurrent breast mass. The surgeon found only necrotic tissue where the mass had been. The surgeon also removed the right axillary nodes that had been involved with tumor. Pathology review of the breast mass and node showed that all malignant cells had been eliminated, showing a cure down to the level of pathological analysis.

Since it is expected that there will not be dose limiting toxicity as is the case with many forms of chemotherapy such as adriamycin or methotrexate, repeated treatments can be applied without significant cumulative injury. Whether this is for primary, metastatic or recurrent lesions, this method can be employed with the usual considerations of PDT that one may need to be careful if the lesions encroach on blood vessels as bleeding could occur when the tumor undergoes necrosis.

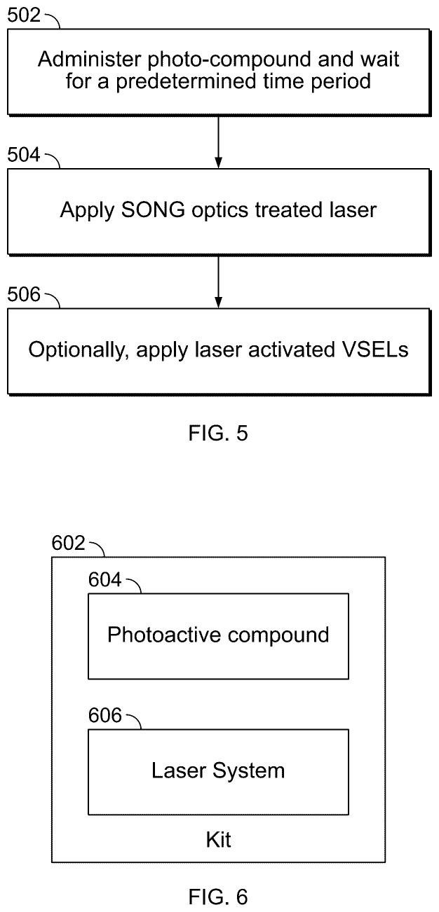

FIG. 5 is a flow chart illustrating another exemplary process using Methylene Blue, in accordance with some embodiments of the present specification. At step 502, a PDT is initiated, comprising administration of Methylene Blue. Methylene blue is administered orally, or using IV routes, and is preferred for oral administration due to its high level of oral absorption. An hour-long waiting period is provided during which the photo-active compounds of Methylene Blue reach the cells, and normal cells that have sufficient aerobic energy reject the photo-active compound, while the neoplastic cells that are abnormal and have low energy are unable to reject the photo-active compound.

At step 504, a laser beam is transmitted through the SONG optical device in accordance with the present specification, to apply light of wavelength of 660 nm (red laser light) with a primary beam power of 500 mW, to stimulate a photo-oxidation reaction. The wavelength of 660 nm is within the peak absorption band for causing photo-oxidation of Methylene Blue. The selection of the photo-active compound influences the selection of energy of source laser beam, the power of the source laser beam, and the wavelength of the source laser beam, to achieve the desirable outcome. The method of FIG. 5 achieves a penetration depth of light that is at least 10% to 3000% more than conventional PDT methods. The depth of penetration ensures that enough bulk photons reach and result in oxidation of neoplastic cells.

At step 506, optionally, laser activated VSELs are used to supercharge NKCs and the immune system to clean-up the remaining neoplastic cells, while still leaving the normal cells unharmed.

Use Case with PDT and SONG Laser in Treatment of Prostate Neoplasia

In an exemplary use case where methylene blue was used, a 68-year-old man with prostate neoplasia and a PSA of 16 was given 50 mg of methylene blue orally and also intra-rectally. After an hour to concentrate in the neoplastic cells in prostate tissue and clear from normal cells, a 500 mW primary power SONG optics device treated laser was passed through a beam expander and a SONG Generator device with a measured peak output of 200 mW and adjusted to reduce the output 60% from the peak power to an 80 mW ultimate output through the device. The treated laser beam was directed at the prostate bed through the perineum for 10 minutes. A few weeks later the patient's PSA had reduced by 50% to a level of 8 with no adverse effects along with improvements in urinary symptoms. The case described here shows that other less penetrating wavelengths may be used for pathological lesions closer to the surface of the body, whereas deeper penetration is required for reaching to lesions situated deep within the skin surface.

Embodiments of the present specification demonstrate that different pairings of photo-active compounds with the respective appropriate wavelength lasers also produce a significant anti-neoplastic effect, usually with few or no side effects. While exemplary, these two methods (using Photofrin® and Methylene Blue) of combining a SONG Laser device with a photo-active compound to extend the range of depth of lesions that can be effectively treated, indicate that any combination of a photoactive compound and a SONG Laser device could be configured in a wavelength range from ultraviolet through visible through infrared wavelengths to achieve intended anti-neoplastic effects beyond those that were previously possible.

FIG. 6 is a block diagram showing a kit which includes at least one photoactive compound and a laser system, in accordance with some embodiments of the present specification. Kit 602 includes at least one photoactive compound 604. Photoactive compound 604 can be a dye, Photofrin, Indocyanine Green, Methylene Blue, or any other compound suitable for the photoactive application of the present specification. Additionally, kit 602 includes a laser system 606 which is adapted to emit laser light. The laser light has a wavelength in a range of 300 nm to 1000 nm. In one embodiment, the wavelength of the laser light is 810 nm. In another embodiment, the wavelength of the light is 660 nm. In embodiments, laser system 606 is a SONG laser system. Laser system 606 is configured to pass a laser beam through a phase cancellation optical element such that the phase cancellation optical element forms a pattern of constructive interference nodes and destructive interference nodes. The phase cancellation optical element comprises a first diffraction grating, a refractive element and a second diffraction grating positioned in series. In embodiments, laser system 606 is configured to form a Fresnel zone in the laser beam. In embodiments, the laser beam generated by system 606 has a power in a range of 3 W to 7 W, and has a power of approximately 5 W in one embodiment.

Additionally, the laser beam generated by system 606 achieves an increased depth of penetration after the administration of photoactive compound 604, when compared to a depth of penetration of the laser beam without the administration of photoactive compound 604. Laser system 606 is configured to form the laser beam such that the depth of penetration of the laser beam in the patient is increased by a factor of 10% to 3000% after the administration of photoactive compound 604 as compared to the scenario without the administration of photoactive compound 604.

In embodiments, the kit may be used to treat a predefined disease in a patient characterized by a neoplastic malignancy, in accordance with some embodiments of the present specification.

In addition, embodiments of the present specification can be applied as a prophylactic measure or treatment. Generally speaking, a tumor growth usually occurs at the primary tumor site. Therefore, in an embodiment, the site at which a tumor primarily occurred may be treated periodically to prevent a tumor recurrence. In the case of a metastatic lesion, the site of the metastases can be treated. In embodiments, if a tumor is removed from a portion of an organ (such as the liver), the entire organ can be treated prophylactically to prevent a tumor occurring on another portion of that organ.

In embodiments, the methods, systems, and compounds described in the present specification can be used to eliminate harmful viruses.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method of treating a predefined disease in a patient characterized by a neoplastic malignancy, the method comprising:
administering a photoactive compound to the patient;
waiting for a first period of time; and
immediately after the first period of time, applying modulated pulses of a laser light beam having a predefined wavelength to an area of the patient for a second period of time, wherein the modulated pulses of the laser light beam are formed by passing the laser light beam from the laser through a phase cancellation optical element, wherein the phase cancellation optical element is adapted to form a pattern of constructive interference nodes and destructive interference nodes, and wherein a depth of penetration of the laser light beam in the patient is increased after the administration of the photoactive compound to the patient relative to a depth of penetration of the laser light beam in the patient without the administration of the photoactive compound.

2. The method of claim 1, wherein the phase cancellation optical element comprises a first diffraction grating, a refractive element and a second diffraction grating positioned in series.

3. The method of claim 1, wherein the laser is configured to form a Fresnel zone in the laser light beam.

4. The method of claim 1, wherein the photoactive compound is a dye.

5. The method of claim 4, wherein the photoactive compound is a porfimer sodium solution.

6. The method of claim 1, wherein the first period of time is in a range of 2 days to 5 days.

7. The method of claim 1, wherein the photoactive compound is nanoscale and microencapsulated Indocyanine Green (ICG).

8. The method of claim 1, wherein the photoactive compound is Methylene Blue.

9. The method of claim 1, wherein a depth of penetration of the laser light beam in the patient is increased after the administration of the photoactive compound to the patient by a factor of 10% to 3000% relative to a depth of penetration of the laser light beam in the patient without the administration of the photoactive compound.

10. The method of claim 1, wherein the laser light beam has a primary beam power of 3 W to 7 W.

11. The method of claim 1, wherein the predefined wavelength ranges from 300 nm to 1000 nm and is dependent on the photoactive compound that is used.

12. The method of claim 1, wherein the predefined wavelength is either equal to or below 400 nm or equal to or above 700 nm.

13. The method of claim 1, wherein the predefined wavelength is 810 nm.

14. The method of claim 1, wherein the predefined wavelength is 660 nm.

15. The method of claim 1, further comprising administering VSEL stem cells to the patient.

16. The method of claim 15, further comprising activating VSEL stem cells prior to said administration by exposing the VSEL stems cells to modulated laser light.

17. The method of claim 15, further comprising activating VSEL stem cells after said administration by exposing the VSEL stems cells in vitro to modulated laser light.

18. The method of claim 1, further comprising reevaluating the patient's neoplastic malignancy to determine an improvement in said malignancy by at least either a reduction in size or a reduction in a rate of growth.

19. The method of claim 18, further comprising repeating the administration of the photoactive compound, waiting for the first period of time, and application of modulated pulses of laser light if the patient's neoplastic malignancy has not shown a reduction in size or rate of growth level of at least 5%.
